(19) United States
(12) Patent Application Publication
(10) Pub. No.: US 2026/0262615 A1
(43) Pub. Date: Sep. 10, 2026

(54) INSECT LARVAE FATTENING MODULE

(71) Applicant: Jesus Francisco SUÁREZ BELLOCH, VALENCIA (ES)

(72) Inventor: Jesus Francisco SUÁREZ BELLOCH, VALENCIA (ES)

(21) Appl. No.: 19/155,114

(22) PCT Filed: Feb. 9, 2024
(86) PCT No.: PCT/ES2024/070073

§ 371 Date: Aug. 8, 2025

(30) Foreign Application Priority Data
Feb. 10, 2023 (ES) P202330099

(51) Int. Cl. A01K 67/36

(52) U.S. Cl. CPC A01K 67/36

(57) ABSTRACT

INSECT LARVAE FATTENING MODULE, formed in a container, with a machinery chamber and another larvae rearing chamber, or in several separate containers that are connected to each other, in which for the rearing a set of longitudinal trays are arranged supported by shelves at different levels, which have folds at their edge, which in addition have a conditioning pipe on their bottom and a heat insulation plate, an air ventilation duct and a channel for temperature and humidity control sensors of the substrate being arranged above each tray, wherein the machinery chamber is open to the outside by means of a micro-perforated plate for the air intake and accommodates the control elements for the automation systems, the valves and of the water and power connections.

### TECHNICAL FIELD

The present invention relates to a fattening module for insect larvae, which is configured in a means of transport such as a large container and equipped with the required means to develop a cultivation and rearing system for insect larvae, allowing said module to be transported according to the user's will to any required farm or location, for the use of adult larvae as animal feed, as well as of fertilizer waste, which is transformed into the feed substrate for the larvae.

It is known in the art that insect larvae need to feed from a moist and nutritive organic substrate with a humidity of 70% and, as they ingest the feed, they release water and break down the substrate particles, so that the substrate finally has a moisture content of less than 30%, which means that the larvae have made good use of the feed to fatten up and become adult larvae.

On the other hand, it is also known that air is a poor conductor of heat and a good conveyor of humidity and, if the difference in temperature and humidity of the substrate is very high compared to that of ambient air, surface crusts may arise on the substrate, which reduce humidity exchange and may prevent ambient ventilation from achieving its objective of gradually removing excess humidity from the substrate.

To mitigate and remedy this effect, the invention aims to improve the fattening module for the larvae by forming a large room for rearing the larvae in the container, which is equipped with climatized and automated mechanical means, where the larvae are placed on their feed substrate at the temperature and humidity required for fattening, which is controlled by devices for regulating the substrate, thereby gradually removing excess humidity from the feed substrate, which in turn prevents surface crusts from forming thereon.

In this manner, the temperature and humidity of the feed substrate for the larvae can be controlled in the rearing module of the container independently of the ventilation and the control of the temperature and ambient humidity inside the container.

### BACKGROUND

In the current state of the art, it is already known that the larvae are fattened in small plastic boxes, which are stacked on top of each other on high shelves arranged in industrial premises that maintain an ambient temperature and humidity suitable for rearing the larvae, but no information about the temperature and humidity of the feed substrate placed inside the boxes for fattening the larvae is made available.

This type of cultivation has the drawback that the ambient temperature and humidity in the industrial premises may not be suitable for the feed substrate arranged in the boxes employed to fatten the larvae, since the initial humidity of the substrate in the boxes needs to be gradually reduced by a high percentage, such as 40% humidity, for the larvae to become adult and be fed properly.

In addition, the boxes stacked on the shelves need to be moved by forklift trucks for them to be controlled, so more staff is required than with the rearing module of the container of the invention, which is fully automated.

### SUMMARY

The present invention intends to provide the market with a fattening module for insect larvae, formed in at least one large container, the interior of which has been equipped with climatized and automated supporting means to place the larvae on a feed substrate, with the temperature and humidity suitable for their fattening controlled by means of regulating devices arranged in a machine room, by means of which excess humidity can be removed little by little from the feed substrate for an improved rearing of the larvae.

In this manner, in the fattening module for insect larvae of the invention, the temperature and humidity of the feed substrate for the larvae can be controlled, while the ventilation and ambient temperature, humidity and carbon dioxide inside the rooms or chambers of the container are controlled.

### DESCRIPTION

According to the invention, the fattening module for insect larvae is formed in a large space or room, the interior of which has been equipped with the required means for rearing the larvae.

To this effect, the invention has at least one container in the interior of which two differentiated working chambers are formed, one chamber for rearing the larvae and another chamber for the machinery.

Said two differentiated chambers can be formed in the same container or in separate containers, i.e. there could be a container for the machinery and one or several containers for the larvae rearing, all of them being connected to that of the machinery.

In the chamber or containers for rearing the larvae, a set of fixed and continuous longitudinal trays are mounted supported by shelves at different levels, which have folds at their edge to prevent larvae from escaping, which in addition have a hot or cold water conditioning pipe on their bottom and a heat insulation plate extending on the outside of the bottom or base thereof.

All of which is arranged such that the trays receive the feed substrate and the larvae to be reared, to which is added an air ventilation duct for a controlled drying of the substrate and a channel for the cabling of temperature and humidity control sensors of the substrate arranged above each tray.

According to the invention, it is provided that air can flow into the machinery chamber and/or into the machinery container through a micro-perforated plate placed on a wall of the chamber for the machinery or container of said machinery to permit adequate functioning of the aerothermal device that has both a heating and cooling power.

Likewise, the machinery chamber or the machinery container accommodates the control elements for the automation systems, the valves and of the water and power connections as well as the electrical panel.

According to the invention, it is provided that the adult larvae and fertilizer waste are evacuated from the trays by means of a pneumatic suction system, which will discharge through a flexible nozzle into a hopper or a tank outside the container.

Furthermore, it is provided according to the invention that a conditioning pipe surrounded by a heat insulation can be arranged on the bottom of the fixed and continuous longitudinal trays supported by the shelves at different levels, so that the bottom of the tray is flat, enabling a movable mat made of a canvas or the like, this being a sufficiently strong fabric, to be arranged on said bottom, so that the larvae cannot be fed therefrom.

A ventilation duct and a channel for the cabling of sensors for controlling the temperature and humidity of the feed substrate for the larvae on the mat are also arranged above each tray.

All of which is arranged such that the substrate for feeding the larvae can be received on the mat and said mat can move longitudinally over the tray by means of rails arranged on the vertical sides of the trays to facilitate the evacuation of the adult larvae and fertilizer waste through an open end of the tray, where a means for folding away the mat during the evacuation is arranged.

According to the invention, it is also provided that the bottom of the fixed and continuous longitudinal trays can be inclined towards a lateral of the tray, where a longitudinal cavity is formed for accommodating a worm screw.

A conditioning pipe of the tray, also covered by a heat insulation plate, is arranged on the outside of the inclined bottom of these trays with the worm screw.

A ventilation duct and a channel for the cabling of sensors for controlling the temperature and humidity of the feed substrate for the larvae on the inclined bottom are arranged above each tray.

Said worm screw is arranged such that it conveys the already adult larvae and fertilizer waste through the trays to a lower discharge pipe of the trays.

### BRIEF DESCRIPTION OF THE DRAWINGS

For a better understanding of the above description, a set of drawings is attached to this specification showing the object of the invention, without the aforementioned graphical representation being meant to constitute a limitation to the specific features of this application.

FIG. 1. Represents a perspective view of a container equipped to form a fattening module for the insect larvae. The interior of the container is divided into two different chambers, one chamber destined to machinery and another chamber destined to larvae rearing, in which stacks of longitudinal trays are arranged supported by shelves at different levels.

FIG. 2. Shows a perspective view of a container equipped to accommodate the machinery required by the automation systems and climatization, water and power control elements therein, which is connected to three containers equipped with longitudinal trays for the fattening and rearing of insect larvae.

FIG. 3. Illustrates a plan view of the equipped container in a fattening module for the insect larvae. The larvae rearing chamber is formed at the inlet of the container, in which chamber longitudinal trays are mounted supported by shelves at several levels and occupy the laterals of the chamber, wherein conditioning pipes are installed on the bottom thereof. A pneumatic suction system is mounted over the trays for the evacuation of the adult larvae and fertilizer waste from the trays, which are discharged through a flexible nozzle flexible into a hopper outside the rearing chamber. Said chamber is connected with a second machinery chamber, where the control elements of the automation systems, as well as the valves and the water and power connections and the electrical panel are accommodated.

FIG. 4. Represents a cross section of the container through the rearing chamber for the insect larvae. In this figure, a closed compartment or chamber can be observed having on each side a stack of “U” shaped section longitudinal trays for the larvae rearing which are supported by shelves at different levels and have on their bottom a conditioning pipe and a heat insulation plate being coupled through the outside of the bottom. A closed door can be seen on the bottom of the chamber being connected to the machine chamber.

FIG. 5. Shows a section detail of a stacking of the trays in which the larvae are to be reared with the feed substrate. It can be seen in this figure that the stacking comprises several parallel trays arranged at different levels and which are supported by shelves, the bottom of all of them having a cold or hot water pipe and a heat insulation encompassing their entire base over the outside thereof.

FIG. 6. Illustrates a section detail of a tray for fattening the insect larvae. It can be observed in this figure that the longitudinal tray has a “U” shaped section and folds at its edges and the bottom of which features a conditioning pipe, while a heat insulation plate is arranged on the outside of the bottom, and a ventilation duct and a channel for the cabling of sensors are arranged above the trays.

FIG. 7. Represents a plan view of another equipped container in a fattening module for the insect larvae. The larvae rearing chamber is formed at the inlet of the container, in which the longitudinal trays have a movable mat on which the larvae are to be reared and, when these have already become adult larvae, they will be able to be evacuated together with the fertilizer waste by the movement of the mat towards the open end of the trays. Said rearing chamber is connected with a second machinery chamber, where the control elements of the automation systems, as well as the valves, the water and power connections and the electrical panel are accommodated.

FIG. 8. Shows a cross section of the container through the rearing chamber for the insect larvae. In this figure, a closed compartment or chamber can be observed having on each side a stack of “U” shaped section longitudinal trays for the larvae rearing, which have on their bottom a heat insulation covering the conditioning pipe to achieve a flat bottom for a movable canvas mat to be placed thereon, which supports the larvae and the feed substrate. A closed door can be seen on the bottom of the chamber being connected to the machine chamber.

FIG. 9. Illustrates a section detail of a stacking of the trays in which the larvae are to be reared with the feed substrate. It can be seen in this figure that the stacking comprises several parallel trays arranged at different levels and which are supported by shelves, the bottom of all of them having the movable canvas mat able to move longitudinally over the tray by means of rails arranged on the vertical sides of the trays.

FIG. 10. Represents a section detail of a tray for fattening the insect larvae. It can be seen in this figure that the longitudinal tray has a “U” shaped section with folds at its edges and the bottom of which has a heat insulation plate inside which the conditioning pipe is accommodated to achieve a flat bottom for the movable mat to be placed thereon, which can move longitudinally over the tray to evacuate the adult larvae and the fertilizer waste from the trays, and a ventilation duct and a channel for the cabling of sensors are arranged above the trays.

FIG. 11. Shows a plan view of another equipped container in a fattening module for the insect larvae. The larvae rearing chamber is formed at the inlet of the container, in which the longitudinal trays have a bottom inclined towards a lateral of the tray, where a longitudinal cavity is formed for accommodating a worm screw. Said rearing chamber is connected with a second machinery chamber, where the control elements of the automation systems, as well as the valves, the water and power connections and the electrical panel are accommodated.

FIG. 12. Illustrates a cross section of the container through the rearing chamber for the insect larvae. In this figure, a closed compartment or chamber can be observed having on each side a stack of longitudinal trays for the larvae rearing and a bottom inclined towards a lateral of the tray, where a longitudinal cavity is formed for accommodating a worm screw, and the conditioning pipe covered by the heat insulation plate is arranged on the outside of the bottom.

FIG. 13. Represents a section detail of a stacking of the trays in which the larvae are to be reared with the feed substrate. It can be seen in this figure that the stacking comprises several parallel trays arranged at different levels which are supported by shelves, all of them having a bottom inclined towards a lateral of the tray, where a longitudinal cavity is formed for accommodating a worm screw, which is arranged such that, as it rotates, the adult larvae and fertilizer waste are conveyed to an end of the tray at which a lower discharge pipe is formed for the collection thereof.

FIG. 14. Shows a section detail of a tray for fattening the insect larvae. It can be observed in this figure that the longitudinal tray has folds at is edges and a bottom inclined towards a lateral of the tray, where a longitudinal cavity is formed for accommodating a worm screw, which is arranged such that, as it rotates, the adult larvae and fertilizer waste are conveyed to an outlet pipe leading out of the trays for the collection thereof, and a ventilation duct and a channel for the cabling of sensors are arranged above the trays.

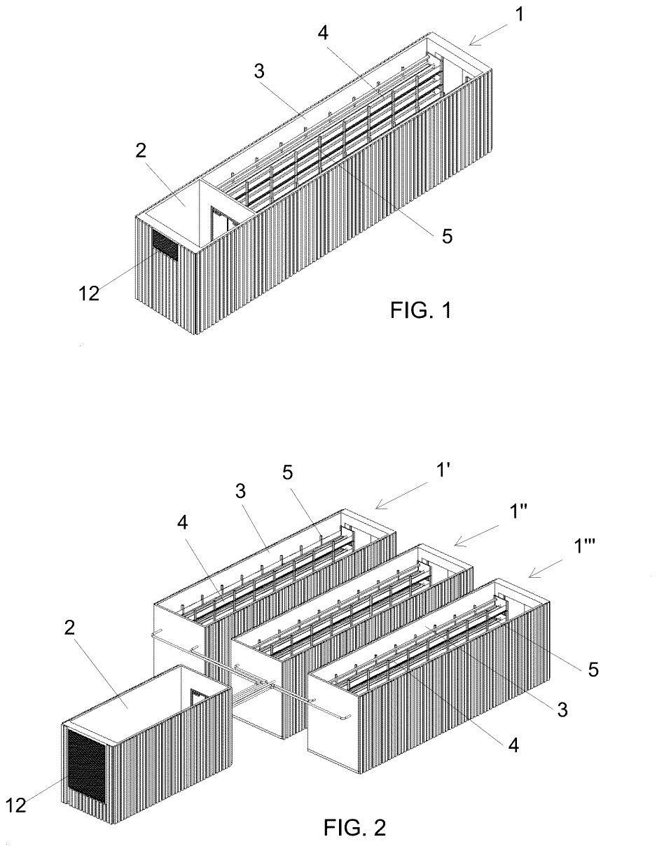

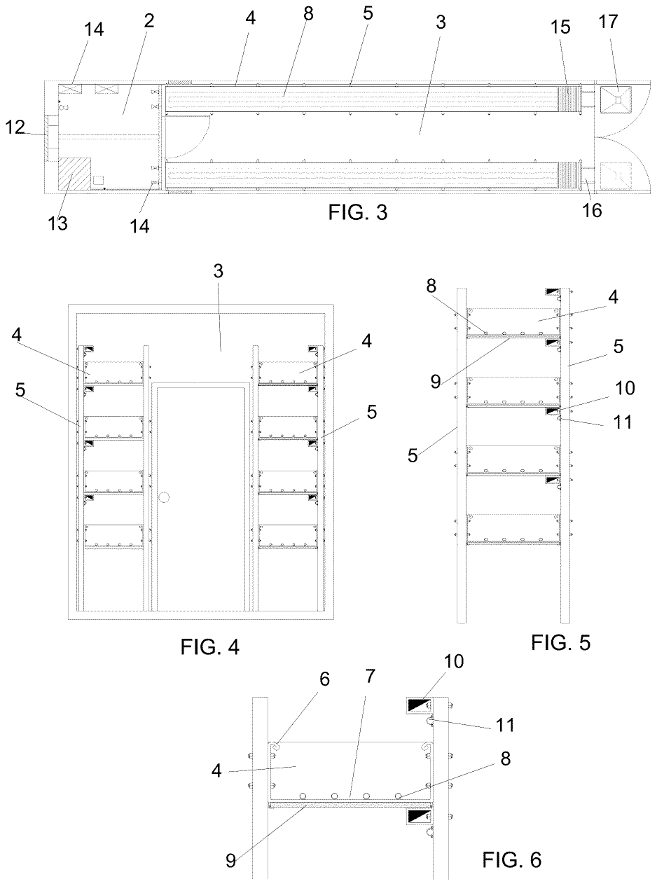

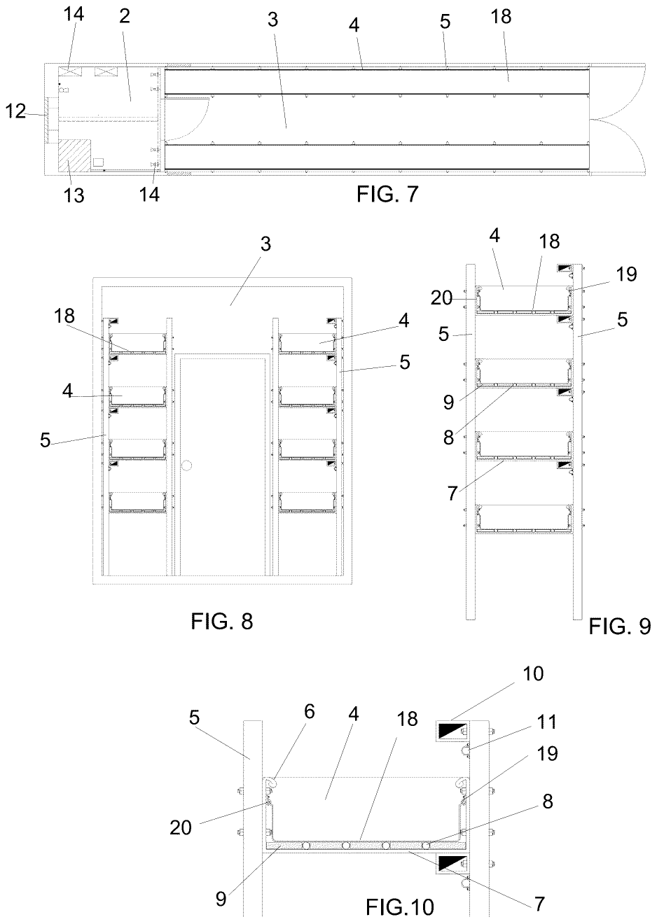

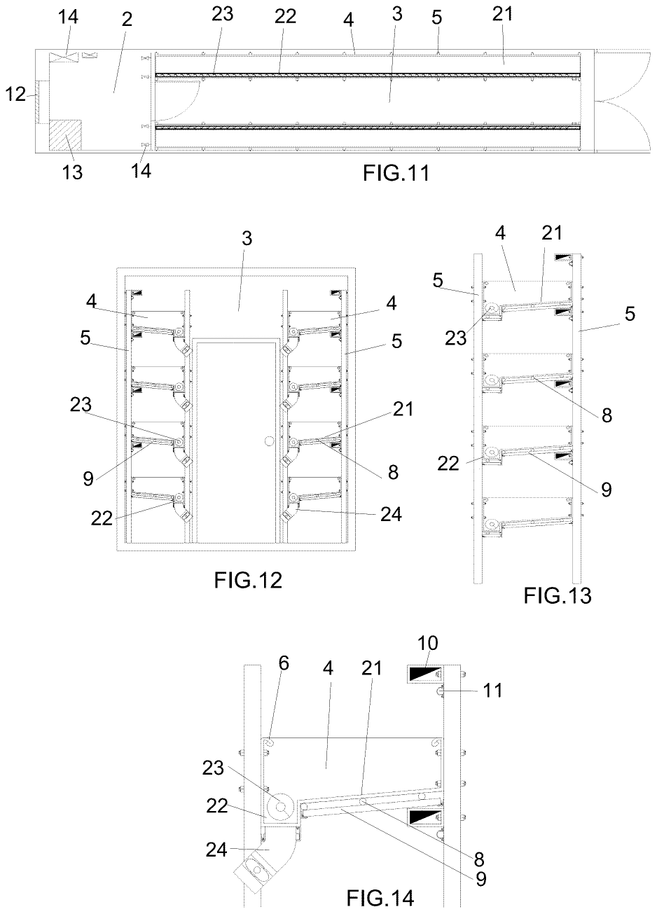

### DETAILED DESCRIPTION

The different components the fattening module for insect larvae is composed of according to a claimed embodiment are described in detail and numbered here below.

According to the invention, the fattening module for insect larvae is formed in a large space or room, the interior of which has been equipped with the required means for rearing the larvae.

In the first figure, it can be observed that the module is formed inside at least one container (1), in which two differentiated working chambers are arranged, one machinery chamber (2) and one larvae rearing chamber (3).

In the first and second figures, it can be observed that both differentiated chambers can be formed in the same container (1) or in separate containers (1′), (1″), (1′″), i.e. there could be several containers just with larvae rearing chambers (3), all of them being connected to a container with a machinery chamber (2).

In the first, second, third and fourth figures, it can be observed that a set of fixed and continuous longitudinal trays (4) are mounted in the larvae rearing chamber (3) being supported by shelves (5) at different levels.

Furthermore, in the fourth, fifth and sixth figures, it can be seen that the trays have folds (6) at their edge, to prevent larvae from escaping, which in addition have a hot or cold water conditioning pipe (8) on their bottom (7) and a heat insulation plate (9) extending on the outside of the bottom (7) or base thereof.

In the fourth, fifth and sixth figures, it can be observed that an air ventilation duct (10) for drying the substrate and a channel (11) for the cabling of sensors for controlling the temperature and humidity of the substrate are arranged above each tray.

All of which is arranged such that the trays receive the feed substrate for the larvae to be reared, to which is added the required means for controlling the temperature and humidity of the substrate so as to improve the larvae feeding and fattening.

In the first, second and third figures, it can be also observed that the machinery chamber or container (2) is open to the outside by means of a micro-perforated plate (12) for the air intake to permit adequate functioning of an aerothermal device (13) that has a high heating and cooling power.

In the third figure, it can be observed that the control elements of the automation systems, the valves and of the water and power connections as well as the electrical panel (14) are accommodated in the machinery chamber (2).

Likewise, it can be observed in said third figure that the adult larvae and fertilizer waste are evacuated from the trays by means of a movable pneumatic suction system (15), above the trays, which discharges through a flexible nozzle (16) into a hopper (17) or tank outside the container.

In the seventh, eighth, ninth and tenth figures, it can be seen that the conditioning pipe (8) covered by a heat insulation (9) can be arranged on the bottom (7) of the longitudinal trays (4) supported by the shelves (5) at different levels, so that the bottom (7) of the tray is flat to arrange a movable strong fabric or canvas mat (18) thereon.

Said mat is arranged to be able to receive the feed substrate with the larvae and able to move longitudinally over the tray by means of rails (19) arranged on the vertical sides (20) of the trays.

All of which is arranged such that the evacuation of the adult larvae and fertilizer waste through an open end of the tray is facilitated, where a means for folding away the mat is provided.

In the eleventh, twelfth, thirteenth, and fourteenth figures, it can be observed that the bottom (21) of the fixed and continuous longitudinal trays (4) can be inclined towards a lateral of the tray, where a longitudinal cavity (22) is formed for accommodating a worm screw (23).

In said figures, it can be also seen how the conditioning pipe (8), which is covered by a heat insulation plate (9), is arranged on the outside of the inclined bottom.

All of which is arranged such that the worm screw conveys the adult larvae and fertilizer waste to a lower discharge pipe (24) of the trays.

What is claimed is:

1. INSECT LARVAE FATTENING MODULE, formed in a large space or room, the interior of which has been equipped with the required means for rearing the insect larvae, characterized in that the module is formed inside at least one container (1), wherein two differentiated chambers are arranged within the same container (1) or in separate containers (1′), (1″), (1′″) that are connected to each other and which are arranged to form a machinery chamber or container (2) and another larvae rearing chamber or container (3), in which a set of fixed and continuous longitudinal trays (4) are arranged supported by shelves (5) at different levels, which have folds (6) at their edge to prevent larvae from escaping, which in addition have a hot or cold water conditioning pipe (8) on their bottom (7) and a heat insulation plate (9) extending on the outside of the bottom, all of which is arranged such that the trays receive the feed substrate and the larvae to be reared, to which is added an air ventilation duct (10) for drying the substrate and a channel (11) for the cabling of temperature and humidity control sensors of the substrate arranged above each tray, wherein the machinery chamber (2) is open to the outside by means of a micro-perforated plate (12) for the air intake to permit adequate functioning of a heating and cooling aerothermal device (13) and accommodates the control elements for the automation systems, the valves and of the water and power connections as well as an electrical panel (14).

2. INSECT LARVAE FATTENING MODULE, according to the first claim, characterized in that the adult larvae and fertilizer waste are evacuated from the trays by means of a movable pneumatic suction system (15), which discharges through a flexible nozzle (16) into a hopper (17) or tank outside the container.

3. INSECT LARVAE FATTENING MODULE, according to the first claim, characterized in that the conditioning pipe (8) covered by a heat insulation (9) can be arranged on the bottom (7) of the fixed and continuous longitudinal trays (4) supported by the shelves (5) at different levels, so that the bottom of the tray is flat, enabling a movable strong fabric or canvas mat (18) to be arranged thereon, on which the feed substrate with the larvae can be received and which is able to move longitudinally over the tray by means of rails (19) arranged on the vertical sides (20) of the trays facilitating the evacuation of the adult larvae and fertilizer waste through an open end of the tray, where a means for folding away the mat is arranged.

4. INSECT LARVAE FATTENING MODULE, according to the first claim, characterized in that the bottom (21) of the fixed and continuous longitudinal trays (4) can be inclined towards a lateral of the tray, where a longitudinal cavity (22) is formed for accommodating a worm screw (23), the conditioning pipe (8) covered by a heat insulation plate (9) being arranged on the outside of the inclined bottom, all of which is arranged such that the worm screw conveys the adult larvae and fertilizer waste to a lower discharge pipe (24) of the trays.
